(19) United States
(12) Patent Application Publication
(10) Pub. No.: US 2006/0021613 A1
(43) Pub. Date: Feb. 2, 2006

(54) Accessory for nebulizer inhaler system

(76) Inventor: Robert Overlander, Nampa, ID (US)

Correspondence Address: PEDERSEN & COMPANY, PLLC, P.O. BOX 2666, BOISE, ID, 83701, US

(21) Appl. No.: 10/910,014

(22) Filed: Aug. 2, 2004

(51) Int. Cl. A61M 11/00; A61M 15/00

(52) U.S. Cl. 128/200.14; 128/203.12; 128/203.16

(57) ABSTRACT

An accessory for an inhaler unit may be retrofit into a conventional nebulizer system having a manifold or an inlet air tube. The accessory includes a one-way air valve that closes except when air is being inhaled. The one-way valve closes to prevent exhaled air/gas from flowing through the manifold and inlet air tube and out to the surrounding atmosphere. Thus, exhaled air/gas does not flow out of the system and does not carry medicine with it, greatly reducing medicine waste. Because the preferred inhaler system does not include any exhale/exhaust valve or any other outlet for exhaled air/gas, the user may loosen his lips, for example, to exhale. The accessory may be provided simply as a single one-way valve retrofit into an existing air tube of a conventional nebulizer system, as an air tube-valve combination that may be attached to a nebulizer system manifold, or as original equipment manufacture (OEM) for a nebulizer system.

### BACKGROUND OF THE INVENTION

1. Field of the Invention

The present invention relates to an inhaler attachment device that is used in combination with a conventional inhaler system, commonly known as a nebulizer. More specifically, the present invention relates to a one-way valve system for preventing the loss of medication during exhalation.

2. Related Art

Many patients with acute or chronic lung disease, such as asthma, emphysema, bronchitis, or pneumonia, use medication delivered in an aerosol form via a nebulizer. Most nebulizers deliver medication continuously throughout the patient's respiratory cycle. Yet, the patient only receives the medication in their lungs during inhalation. Therefore, when the patient is exhaling, the instant inventor believes that considerable medication is lost to the atmosphere. For elderly patients surviving on Medicare and Social Security, the loss of this medication can mean a loss of a considerable amount of money. Therefore, the instant inventor believes that there is a need for an inexpensive solution to prevent the loss of medication to the atmosphere during exhalation.

Issued patents relating to inhalers and inhaler valves are reviewed hereinafter.

Kocinski (U.S. Pat. No. 5,170,782) discloses an improved aerosol chamber for an ultrasonic nebulizer which prevents relatively large amounts of medicament droplets from being inhaled by a patient. Kocinski provides a geyser baffle within a mist tube that directs large droplets away from the mist tube slot. The mist tube and chamber housing create a cyclone effect which is effective to cause larger droplets to condense and return to the fluid reservoir before they can be delivered to the patient.

Radcliff et al. (U.S. Pat. No. 5,813,401) discloses a shuttle cylinder with check valve that slides within the breath tube. When the shuttle is at the end of the tube toward the patient, the shuttle permits the mist to reach the patient. When the patient exhales, the shuttle moves to the opposite end of its travel and closes off the supply of mist while permitting the exhaled air to exit. Radcliff's nebulizer control valve is aimed at permitting the flow of medication only when the patient is actually inhaling the medicated aerosol to help prevent the waste of medication and minimize the exposure of others to the medication.

Bienvenu et al. (U.S. Pat. No. 6,412,481) discloses a one-way valve for admitting ambient air on one end of the elongated conduit in combination with a calibrated pressure exhalation valve. The calibrated pressure exhalation valve is spring-loaded and allows for various calibrations. When the user inhales, air is admitted into the conduit and into the user's respiratory system. As the user exhales, the one-way valve prevents escape of exhaled gas through the first end of the conduit and diverts it to the calibrated pressure exhalation valve, which is provided with an outlet port. Once the pressure in the conduit exceeds a pre-determined setting, the exhalation gas is allowed to escape into the atmosphere through the calibrated pressure exhalation valve. Bienvenu's conduit is aimed a building up positive pressure in the user's sealed airways to keep the airways distended for more effective delivery of the medication.

O'Rourke (U.S. Pat. No. 6,659,100) discloses a peep valve mounted inside a hollow body, and a pre-set compression spring mounted between the valve and an adjustable cap or cam lever in the main body of the device. O'Rourke teaches a pair of valve openings extending through the main wall of the body, one for inhalation and one for exhalation. O'Rourke's device is aimed at generating a positive backpressure in the airways of the user so as to keep the airways open and restore normal breathing.

Despite the large number of inhalation valve systems, none of the above-mentioned devices offer a simple solution for eliminating the waste of medication. The inlet valve systems disclosed in the prior art above must operate in communication with a more complicated outlet valve that is hard to manufacture and difficult to clean. Therefore, the inventor believes that there is still a need for a simple accessory including a valve system for inhalers.

### SUMMARY OF THE INVENTION

The present invention comprises an inhaler attachment device that is an accessory for use with a conventional inhaler system, commonly known as a nebulizer. The accessory is adapted to reduce waste of the medication via a device that is simple to manufacture, inexpensive to purchase, and easy to use. Preferably, the device adds a single one-way valve to a normal air/inlet tube of the inhaler system. The one-way valve may comprise a flapper operating inside the inlet tube or may comprise other structure that allows airflow into the airflow tube, but not out of the tube. Preferably, the resulting device does not have multiple valves, adjustable valves, or other structure that complicates use or cleaning of the system.

The preferred embodiment of the invention comprises a generally cylindrical, hollow, flexible tube that is used in combination with a conventional inhaler, in order to aid a patient suffering from asthma, emphysema, or other respiratory illness. The inhaler typically comprises a manifold, a mouthpiece, and a nebulizer unit (from which the overall inhaler system gains the name “nebulizer”). The manifold may have a body, two openings coaxial to the body, and a third opening that is perpendicular to the longitudinal axis of the body. In the preferred embodiment, the flexible tube may frictionally connect to one of the two openings on the manifold coaxial to the body of the manifold. The mouthpiece connects to the other coaxial opening on the manifold body, and the nebulizer connects to the opening on the manifold that is perpendicular to the longitudinal axis of the manifold body. Preferably, the flexible tube comprises an inlet opening and an outlet opening with the inlet opening comprising a one-way valve for receiving atmospheric air.

Preferably, the one-way valve at the inlet of the tube comprises a flapper and a cap. The flapper preferably comprises a hinge/attachment portion and a preferably cylindrical or elliptical shaped flat portion. The flat portion may be flexible or rigid and a different shape depending on the shape of the tube and/or depending on the shape of the sealing surface with which it cooperates. The preferred cap comprises a cylindrical portion that encloses the outer surface of the tube's inlet opening, and a generally cylindrical opening in the top surface of the cap for allowing air to pass into the tube.

In the preferred embodiment, the hinge/attachment portion on the flapper folds over the outer surface of the tube and is held in position as the cap slides over the outer surface of the tube, pressing/trapping the hinge between the inner surface of the cap and the outer surface of the tube. Therefore, the flat portion of the flapper is normally seated against an interior surface of the top of the cap, preventing airflow through the opening in the cap. As the patient inhales through the mouthpiece, the flapper is pulled toward the user, unseating the flapper, and allowing atmospheric air to enter the tube through the opening in the cap. As the atmospheric air enters the tube, it mixes with the medication from the nebulizer and enters the patient's respiratory system via the mouthpiece. As the patient exhales, the exhaled air forces the flapper against the cap, so that it blocks the opening in the cap, and prevents medication and air from escaping.

In an alternative embodiment, the one-way valve may comprise a flapper, a cup, and a cap. Preferably, the hinge/attachment portion of the flapper folds over the outer surface of the cup, and as the cup slides into the tube, the hinge is pressed/trapped between the outer surface of the cup and the inner surface of the tube. The cap is then fitted/slid over the outer surface of the tube. Preferably, the cup has a generally cylindrical hole in its “bottom”. The cup may include a filter for filtering the atmospheric air before it enters the tube. The flat portion of the flapper is seated against the bottom of the cup, and therefore, prevents airflow out of the tube. As the patient inhales, atmospheric air enters the tube through the opening in the cap, passes through the opening in the cup, and eventually into the patient's respiratory system. As the patient exhales, the exhaled air forces the flapper against the bottom of the cup, and blocks the opening in the cup, preventing air and medication from escaping into the atmosphere.

Other embodiments of one-way valves and systems for attaching them to the flexible tube are envisioned by the inventor, and will be apparent to one of average skill in the art after reading and viewing this Description and the Drawings.

The present invention may be used with conventional inhaler devices or other respiratory devices. Preferably, the tube and one-way valve parts are realeasably attached to each other and to the manifold for easy cleaning and packaging and replacement of the flapper. Alternatively, these parts may be integrally molded and/or permanently affixed to each other. The preferred one-way valve system is simple in design to facilitate easy construction, and is a simple solution to prevent the loss of medication for those who cannot afford or operate more complex devices.

### BRIEF DESCRIPTION OF THE DRAWINGS

FIG. 1A is a perspective view of one embodiment of the invented inhaler attachment device show assembled with a conventional inhaler/nebulizer.

FIG. 1B is a perspective view of the embodiment shown in FIG. 1A, wherein the conventional inhaler/nebulizer is shown as background in ghost lines.

FIG. 2 is an exploded view of one embodiment of the invented inhaler attachment device.

FIG. 3 is an exploded view of an alternative embodiment of the invented inhaler attachment device.

FIG. 4A is a side view of the assembled embodiment show in FIG. 2, without the filter, wherein the flapper is shown in its inhalation orientation.

FIG. 4B is a side view of the embodiment shown in FIGS. 2 and 4A, wherein the flapper is shown in its exhalation orientation.

FIG. 5A is a side view of the assembled embodiment shown in FIG. 3, wherein the flapper is shown in its inhalation orientation.

FIG. 5B is a side view of the embodiment shown in FIGS. 3 and 5A, wherein the flapper is shown in its exhalation orientation.

### DESCRIPTION OF THE PREFERRED EMBODIMENTS

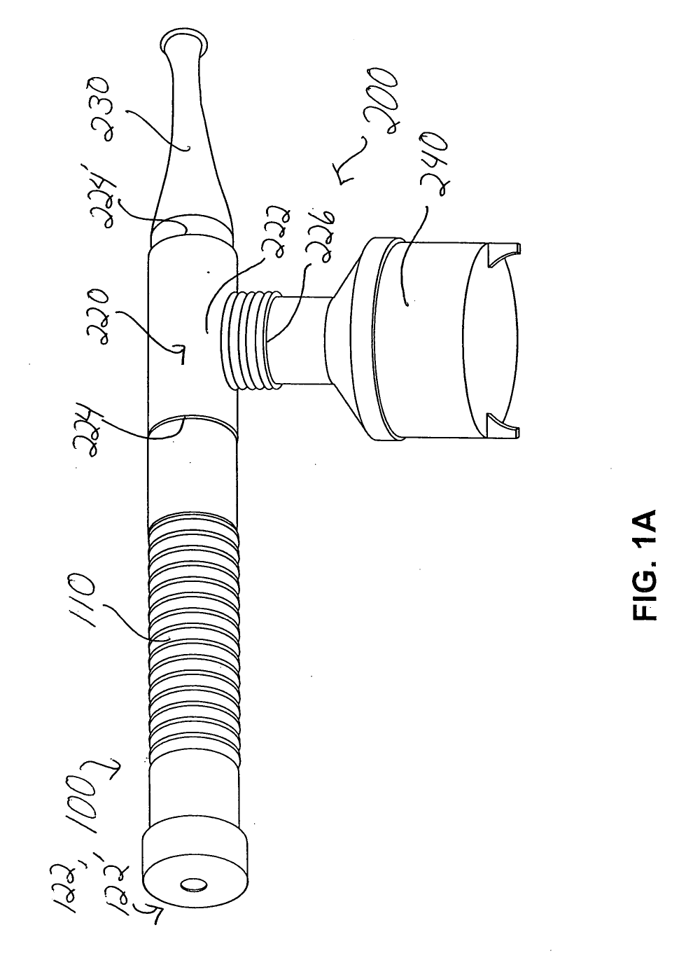

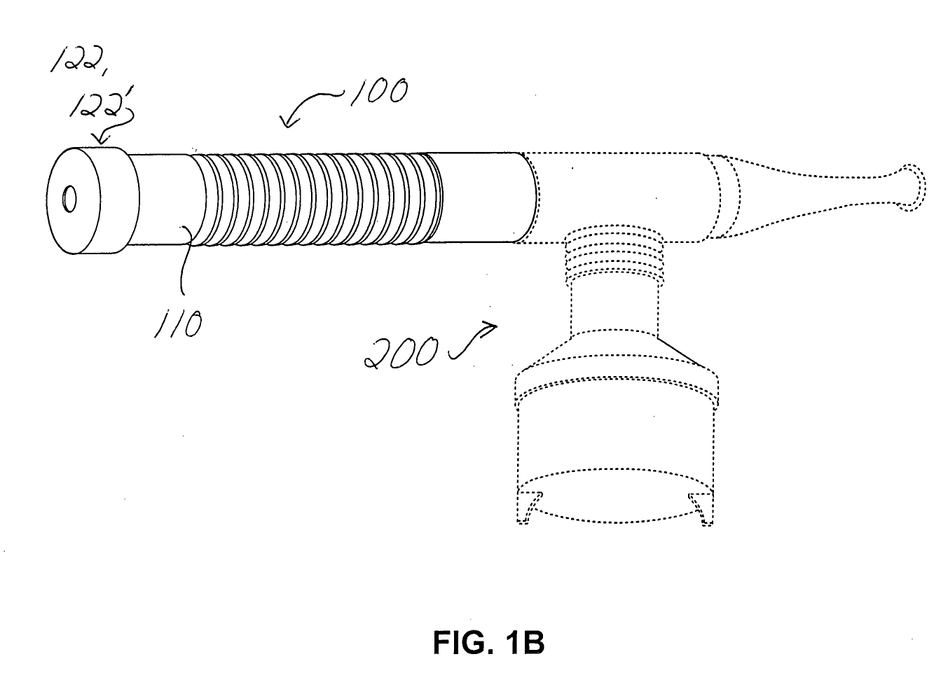

Referring to the Figures, there are shown some, but not the only, embodiments of the present invention, which is referred herein as an inhaler accessory (also referred to as simply “accessory”) for use with a common inhaler/nebulizer. FIG. 1A illustrates one arrangement for attaching the invented inhaler accessory 100 to a conventional inhaler system 200. FIG. 1B illustrates the invented inhaler accessory 100 attached to the conventional inhaler system 200, wherein the conventional inhaler system 200 is shown in ghost lines as background and is not part of the present invention.

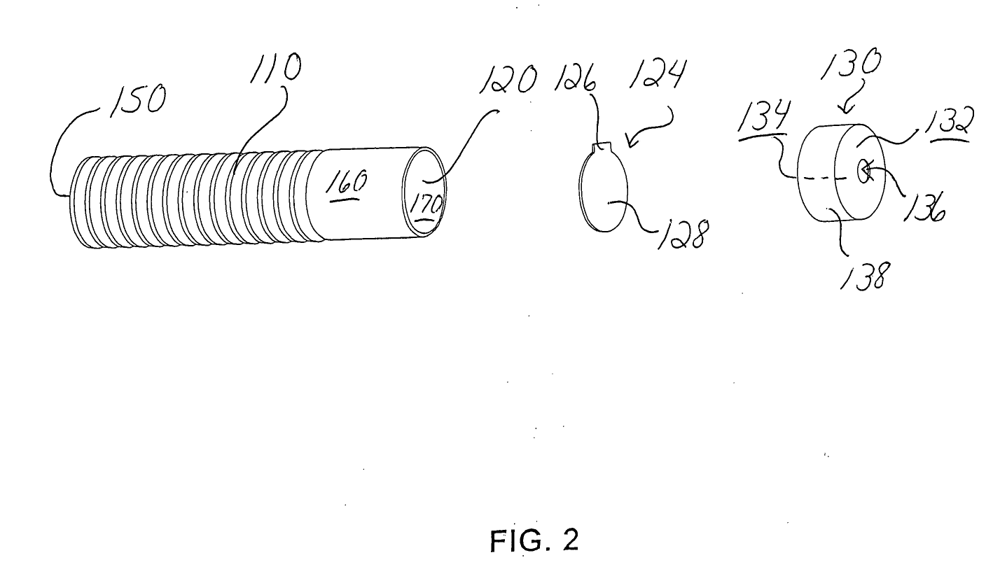

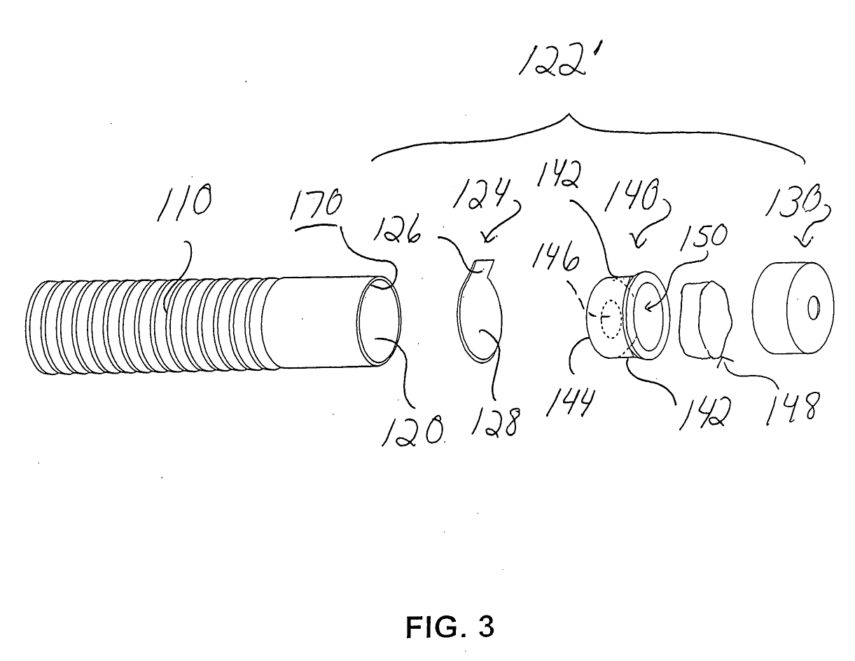

In the preferred embodiment, the accessory comprises a tube 110 having an inlet opening 120 for receiving air, an outlet opening 150 for attachment to a conventional inhaler 200, an outer surface 160, and an inner surface 170 (see FIGS. 2 and 3). Further, the tube 110 preferably at or near the inlet opening 120 comprises a single one-way valve 122 or “check” valve.

Preferably, the conventional inhaler 200 comprises a manifold 220, a mouthpiece 230, and a nebulizer 240 (with “misting cup”). The preferred manifold 220 comprises a body portion 222 having two openings 224, 224′ coaxial to the body portion, and a third opening 226 perpendicular to the longitudinal axis of the body 222. As shown in FIGS. 1A and 1B, the preferred arrangement for connecting the accessory 100 to the conventional inhaler 200, is to connect the outlet opening 150 to one of the coaxial openings 224 on the manifold 220, connect the mouthpiece 230 to the other coaxial opening 224′, and connect the nebulizer 240 to the opening 226 perpendicular to the longitudinal axis of the manifold body 222. Preferably, the accessory 100 is connected to the inhaler 200 via a friction fit allowing the accessory 100 to be easily slid off the manifold 220 for cleaning. Alternatively, other connection methods are possible, such as adhesive, tape, locking mechanisms, or fasteners.

In the preferred embodiment, the one-way valve 122 is preferably a swing check valve in which a flapper 124 comprises a hinge portion 126 and a flat portion 128 (see FIGS. 2 and 3). The flapper 124 pivots between an open position allowing air-flow into the tube 110 and a closed position, preventing air-flow in the reverse direction out of the tube 110. Preferably, the flat portion 128 is flexible, and generally circular or elliptical in shape for easy movement within the tube 110. The flat portion may be other shapes, such as an oval, square, etc. depending on the size or shape of the tube 110 and/or depending on the sealing surface with which it cooperates, so long as its size and shape prevents exhaled air from leaving the tube 110, as well as preventing the flat portion 128 from collapsing into the inlet opening 120. Additionally, the flapper 124 is preferably made of flexible plastic, Mylar™, rubber or other gasket type material, however, other more rigid material may be used, such as plastic, aluminum, etc., as long as the flat portion 128 is capable of being moved on the hinge portion with only minimal air pressure and is adapted to seal with a valve seat.

In the preferred embodiment, the cap 130 comprises a top surface 132, an opening 136 in the top surface 132, an interior surface 134 in the top of the cap, and a cylindrical extension 138 (see FIGS. 2 and 3). Preferably, the cap 130 is made of a rigid plastic for being frictionally fit over the inlet opening 120 of the tube 110. Other rigid or semi-rigid materials may be used for manufacturing the cap 130. Additionally, other methods may be used to secure the cap 130 to the tube 110, such as being adjustable screwed to the tube, adhesive, or fasteners.

Preferably, the hinge portion 126 on the flexible-flapper is folded over the outer surface 160 of the tube 110. The cylindrical extension 138 on the cap 130 is then slid over the outer surface 160 of the tube 110, at the inlet opening 120. The hinge portion 136 is then pressed/trapped between the cylindrical extension 138 and the outer surface 160 of the tube 110. The hinge 136 may be secured to the outer surface of the tube 110 with adhesive or it may be integral with the tube 110. However, it is preferable that the flapper 124 is capable of being removed in order to be cleaned. Preferably, the flat portion 128 of the flapper 124 is seated against the interior surface 134 of the top of the cap, so that it covers the opening 136 in the top of the cap 130.

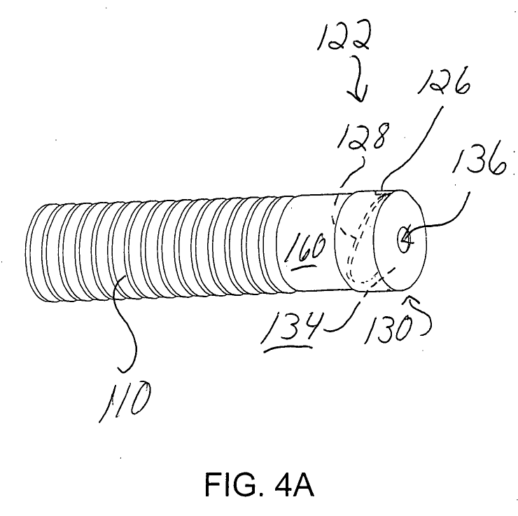

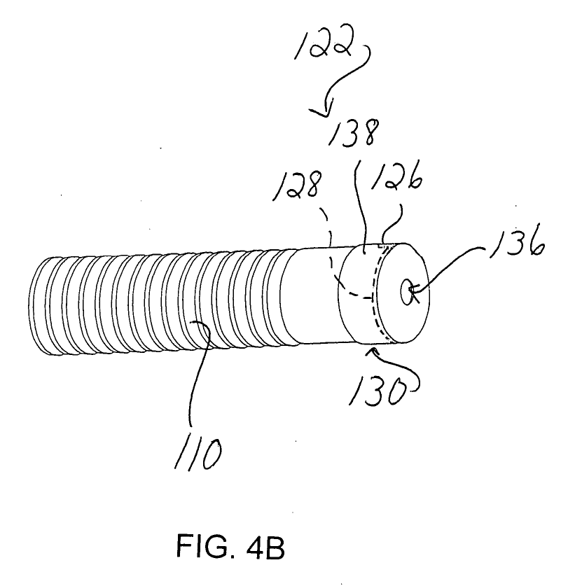

When the patient needs to use the inhaler system 200 they place their lips over the mouthpiece 230. During inhalation, the flat portion 128 of the flapper 124 is pulled away from the interior surface 134 of the top of the cap 130, so that atmospheric air can enter the tube 110 through the opening 136 in the top of the cap 130 (see FIG. 4A). The atmospheric air mixes with the medicine from the nebulizer and is delivered to the patient's respiratory system. Upon exhalation, the pressure of the exhaled air forces the flat portion 128 of the flapper 124 to seal against interior surface 134 of the top of the cap, in turn preventing the exhaled air and medicine from leaving the tube 110 (see FIG. 4B).

Because there is preferably no exhale valve (that is, no valve that opens to allow air/gas exhaled into the mouthpiece and manifold to exit from the system), the user may need to release his/her lips from the mouthpiece to exhale from the mouth, or to exhale from his/her nose. Although this may not be appropriate for some urgent care or other medical cases, it may be an excellent compromise in order to save medicine and money for large numbers of people who use the system in their homes on a daily basis for lung maintenance.

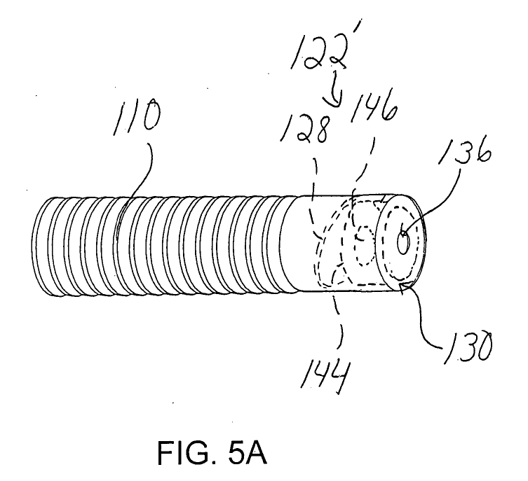

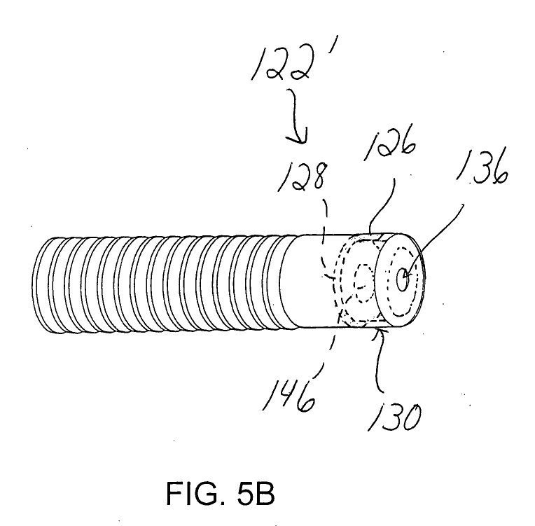

In an alternative embodiment, the one-way valve system 122′ comprises a flexible-flapper 124, a cap 130, and a cup 140 (see FIG. 3). Preferably, the cup 140 comprises a sidewall or axial wall 142, a bottom portion 144, and a circular hole 146 in the bottom of the cup 140. In the alternative embodiment, the hinge 126 is folded over sidewall/axial wall 142 of the cup 140. As the bottom of the cup 144 is slid into the inlet opening 120 if the tube 110, the hinge 126 is pressed/trapped between the sidewall 142 of the cup 140 and the inner surface 170 of the tube 110. The hinge 126 may be fastened or adhered to the sidewall 142 of the cup 140, or it may be formed to be integral with the cup 140. Preferably, the flat portion 128 is seated against the bottom 144 of the cup 140, so that it covers the hole 146 in the bottom of the cup. As the patient inhales, the force of the air pulls the flat portion 128 of the flapper 124 away from the bottom 144 of the cup, so that atmospheric air can pass through the hole 146 in the bottom 144 of the cup (see FIG. 5A). As the patient exhales, the force of the air pushes the flat portion 128 of the flapper 124 toward the bottom 144 of the cup, so that the hole 146 is blocked preventing air and medication from escaping the tube (see FIG. 5B).

A filter may be added to the system to filter incoming air. For example, a filter 148 may be inserted into the interior space 150 of the cup 140 or into another receiving member upstream of the one-way valve. A filter may be installed in other locations in or on the inlet air tube 110, as long as it does not interfere with operation of the one-way valve. The filter may be a pad, sheet or folded sheet of fibrous material, carbon filter contained within close or fibrous material, etc. Multiple of said filters may be supplied to the user for removal and replacement as desired. The filter may be adapted to filter outside bacteria, dander, lint, viruses, or other particles or material, as needed for a particular patient or user.

The preferred accessory may consist only of one one-way valve or “check” valve provided in the conventional inlet/outlet air tube or a nebulizer system. The overall system may consist only of a nebulizer unit, manifold, mouthpiece, and inlet/outlet air tube on one opening of the manifold and including the single one-way valve. Preferably, the overall system has no other one-way valves, and no valves in the manifold or in any member attached to the manifold other than the single one-way valve. Preferably, no adjustable or calibrated valves are included, and the single one-way valve of the invention is not adjustable or calibrated, so it does not operate in any partially open or partially closed position, but simply all-open or all-closed. Other one-way valves besides those drawn may be used, for example a tilting-disk check valve or a lift check valve.

Therefore, the preferred embodiments offer especially in-home, long term users of nebulizer systems a convenient, simple accessory that may be retrofit to their current equipment. They may purchase the inlet air tube with the one-way valve, or they may purchase the one-way valve parts for installation in their current inlet air tube. The system does not require calibration, adjustment, or complex maintenance. It simply requires cleaning as needed or even disposal and refitting with another valve. Thus, by saving substantial amounts of medicine, the system may save hundred of dollars for those on limited incomes.

The terminology “no exhaled air or medicine exits the manifold or the inlet tube” means that no significant amount exits the manifold or the inlet tube, for example, through an exhaust or exhalation valve or tube. With the one-way valve closed and with the user's lips loosened from the mouthpiece, it is possible that a small amount may “leak out” of the manifold via the mouthpiece. This “leakage”, however, is expected to be an insignificant amount and not caused by the force of exhalation but caused merely by the user releasing lips from a mouthpiece having an interior space holding air/medicine.

The flapper may be biased into the closed position by a biasing system such as a spring, flexible plastic, or other biasing means, so that it closes except when the user is inhaling. Alternatively, as in the preferred embodiments, the flapper may merely tend to close upon a slight positive pressure inside the tube relative to outside of the tube such as would be caused upon cessation of inhaling and/or upon a small amount of exhaling (even if the user needs to loosen his lips to exhale).

Although this invention has been described above with reference to particular means, materials and embodiments, it is to be understood that the invention is not limited to these disclosed particulars, but extends instead to all equivalents within the scope of the following claims.

I claim:

1. An inhaler system comprising:
a nebulizer unit;
a manifold connected to the nebulizer unit having a first opening connected to the nebulizer unit, and second and third opposing openings that are generally coaxial and on an axis perpendicular to the first opening, wherein said second opening holds and communicates with a mouthpiece and said third opening holds and communicates with an inlet air tube;
a single one-way valve connected to the manifold, said single one-way valve being provided in the inlet air tube;
wherein said single one-way valve is adapted to open when a user inhales through the mouthpiece, and wherein said single one-way valve is adapted to close when a user exhales through the mouthpiece, so that exhaled air and medicine do not flow out of the inhaler system.

2. An inhaler system as in claim 1, comprising no exhalation valves.

3. An inhaler system as in claim 1, comprising only one valve, which only one valve is said single one-way valve.

4. An inhaler system as in claim 1, wherein said single one-way valve comprises a swing valve.

5. An inhaler system as in claim 1, wherein said single one-way valve comprises a flapper having a flap portion and a hinge portion, the hinge portion being anchored to an outer surface of said inlet air tube.

6. An inhaler system as in claim 5, further comprising a cap on an outer end of said inlet air tube, said cap extending over said outer end and capturing the hinge portion between the cap and the outer surface of the inlet air tube.

7. An inhaler as in claim 6, wherein said cap has an inner radial surface with an aperture for allowing inhaled air into the inlet air tube, and wherein said flap portion seats against said inner radial surface to close-off said aperture when said one-way valved closes.

8. An inhaler system as in claim 1, wherein said single one-way valve comprises a flapper having a flap portion and a hinge portion, the hinge portion being anchored to an inner surface of said inlet air tube.

9. An inhaler system as in claim 8, further comprising a cup member inserted into the outer end of the inlet air tube and adapted to capture said hinge portion between the cup member and said inner surface of said inlet air tube.

10. An inhaler system as in claim 9, wherein said cup member has a bottom radial surface with a cup aperture for allowing inhaled air into the inlet air tube, and wherein said flap portion seats against said bottom radial surface to close-off said cup aperture when said one-way valved closes.

11. An inhaler system as in claim 10, further comprising a cap installed over the outer end of the inlet air tube and over an outer end of the cup member.

12. An inhaler system as in claim 10, wherein said cup member has an interior space and the inhaler system further comprises a filter inside said cup member interior space.

13. An inhaler system as in claim 11, wherein said cup member has an interior space and the inhaler system further comprises a filter inside said cup member interior space between the cup member and the cap.

14. An inhaler system as in claim 1, wherein said single one-way valve is adapted only to open fully upon a user inhaling and to close fully upon cessation of said inhaling.

15. An inhaler system comprising:
a dispensing unit adapted to dispense a medicine into air to be breathed by a patient;
a manifold connected to the dispensing unit and having two opposing and generally coaxial openings;
a mouthpiece connected to one of said openings and an inlet air tube connected to the other of said two openings;
a one-way valve provided in said inlet air tube that is adapted to allow air to be inhaled into the manifold via the one-way valve and the inlet air tube and into the mouthpiece, and the one-way valve also adapted to prevent any air or exhaled air from exiting the inhaler system.

16. An inhaler system as in claim 15, wherein the one-way valve is either fully-open or fully-closed.

17. An inhaler system as in claim 15, comprising no exhalation valves.

18. An inhaler system as in claim 15, comprising only one valve, which only one valve is said one-way valve.

19. An inhaler system as in claim 15, wherein said one-way valve comprises a swing valve.

20. An inhaler system as in claim 15, wherein said one-way valve comprises a flapper having a flap portion and a hinge portion, the hinge portion being anchored to an outer surface of said inlet air tube.

21. An inhaler system as in claim 20, further comprising a cap on an outer end of said inlet air tube, said cap extending over said outer end and capturing the hinge portion between the cap and the outer surface of the inlet air tube.

22. An inhaler as in claim 21, wherein said cap has an inner radial surface with an aperture for allowing inhaled air into the inlet air tube, and wherein said flap portion seats against said inner radial surface to close-off said aperture when said one-way valved closes.

23. An inhaler system as in claim 15, wherein said single one-way valve comprises a flapper having a flap portion and a hinge portion, the hinge portion being anchored to an inner surface of said inlet air tube.

24. An inhaler system as in claim 23, further comprising a cup member inserted into the outer end of the inlet air tube and adapted to capture said hinge portion between the cup member and said inner surface of said inlet air tube.

25. An inhaler system as in claim 24, wherein said cup member has a bottom radial surface with a cup aperture for allowing inhaled air into the inlet air tube, and wherein said flap portion seats against said bottom radial surface to close-off said cup aperture when said one-way valved closes.

26. An inhaler system as in claim 25, further comprising a cap installed over the outer end of the inlet air tube and over an outer end of the cup member.

27. An inhaler system as in claim 25, wherein said cup member has an interior space and the inhaler system further comprises a filter inside said cup member interior space.

28. An inhaler system as in claim 26, wherein said cup member has an interior space and the inhaler system further comprises a filter inside said cup member interior space between the cup member and the cap.

29. An inhaler system as in claim 15, wherein said one-way valve is adapted only to open fully upon a user inhaling and to close fully upon cessation of said inhaling.
